(19) United States
(12) Patent Application Publication
(10) Pub. No.: US 2014/0332007 A1
(43) Pub. Date: Nov. 13, 2014

(54) PATIENT INTERFACE, CUSHION THEREOF, AND MANUFACTURING METHOD

(71) Applicant: KONINKLIJKE PHILIPS N.V., Eindhoven (NL)

(72) Inventors: Dmitry Nikolayevich Znamenskiy, Eindhoven (NL); Ruud Vlutters, Eindhoven (NL); Octavian Soldea, Kiryat-Bialik (IL); Karl Catharina Van Bree, Eindhoven (NL); Franciscus Hendrikus Van Heesch, Eindhoven (NL); Leo Jan Velthoven, Eindhoven (NL)

(21) Appl. No.: 14/364,310

(22) PCT Filed: Dec. 20, 2012
(86) PCT No.: PCT/IB2012/057525

§ 371 Date: Jun. 11, 2014

Related U.S. Application Data
(60) Provisional application No. 61/580,473, filed on Dec. 27, 2011.

(51) Int. Cl. A61M 16/06

(52) U.S. Cl. CPC A61M 16/0622; A61M 16/0666; A61M 2016/0661
USPC 128/206.24

(57) ABSTRACT

A cushion arrangement for a patient interface (for communicating with the nose or the nose and mouth of a patient) comprises a cushion and a shaping structure in contact with the cushion. The shaping the comprises a thermo-shrink material, and the local dimension of themo-shrink material determines a level of local compression or expansion of the cushion. The shaping structure enables the cushion to be customised for the end user.

### FIELD OF THE INVENTION

The present invention relates to patient interfaces for transporting a gas to and/or from an airway of a user, and to a method of manufacturing the same. It also relates to the cushion of such a patient interface.

### BACKGROUND OF THE INVENTION

There are numerous situations where it is necessary or desirable to deliver a flow of breathing gas non-invasively to the airway of a patient, i.e. without inserting a tube into the airway of the patient or surgically inserting a tracheal tube in their oesophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnoea syndrome, in particular, obstructive sleep apnoea (OSA).

Non-invasive ventilation and pressure support therapies involve the placement of a patient interface device including a mask component on the face of a patient. The mask component may be, without limitation, a nasal mask that covers the patient's nose, a nasal pillow/cushion having nasal prongs that are received within the patient's nostrils, a nasal/oral mask that covers the nose and mouth, or a full face mask that covers the patient's face. The patient interface device interfaces between the ventilator or pressure support device and the airway of the patient, so that a flow of breathing gas can be delivered from the pressure/flow generating device to the airway of the patient.

Such devices are typically maintained on the face of a patient by headgear having one or more straps adapted to fit over/around the patient's head.

FIG. 1 shows a typical system to provide respiratory therapy to a patient. This is termed a “patient interface” in the description and claims.

The system 2 includes a pressure generating device 4, a delivery conduit 16 coupled to an elbow connector 18, and a patient interface device 10. The pressure generating device 4 is structured to generate a flow of breathing gas and may include, without limitation, ventilators, constant pressure support devices (such as a continuous positive airway pressure device, or CPAP device), variable pressure devices, and auto-titration pressure support devices.

Delivery conduit 16 communicates the flow of breathing gas from pressure generating device 4 to patient interface device 10 through the elbow connector 18. The delivery conduit 16, elbow connector 18 and patient interface device 10 are often collectively referred to as a patient circuit.

The patient interface device 10 includes a mask 12, which in the exemplary embodiment is a nasal and oral mask covering the nose and mouth. However, any type of mask, such as a nasal-only mask, a nasal pillow/cushion or a full face mask, which facilitates the delivery of the flow of breathing gas to the airway of a patient, may be used as mask 12.

The mask 12 includes a cushion 14 coupled to a shell 15. The cushion 14 is made of a soft, flexible material, such as, without limitation, silicone, an appropriately soft thermoplastic elastomer, a closed cell foam, or any combination of such materials. An opening in shell 15, to which elbow connector 18 is coupled, allows the flow of breathing gas from pressure generating device 4 to be communicated to an interior space defined by the shell 15 and cushion 14, and then to the airway of a patient.

The patient interface device 10 also includes a headgear component 18, which in the illustrated embodiment is a two-point headgear. Headgear component 18 includes a first and a second strap 20, each of which is structured to be positioned on the side of the face of the patient above the patient's ear.

Headgear component 18 further includes a first and a second mask attachment element 22 to couple the end of one of the straps 20 to the respective side of mask 12.

A problem with this type of mask is that the headgear force vectors necessary to achieve a robust and stable seal against the face of the patient can cut a straight line near the corners of a patient's eyes, which can be uncomfortable and distracting.

In order to avoid this, it is well known to include a forehead support to spread the required forces over a larger area. In this way, an additional cushion support on the forehead balances the forces put by the mask around the nose or nose and mouth.

However, the mask may still be uncomfortable. There are many differences between human faces, and it is very hard to develop a limited number of masks that should fit everyone. Customization of masks is the logical solution to this problem, but currently, the associated costs and fabrication time prohibit this.

### SUMMARY OF THE INVENTION

According to the invention, there is provided a cushion arrangement as claimed in claim 1 (for a patient interface), a method of customising a cushion arrangement for a patient interface as claimed in claim 6, and an apparatus as claimed in claim 14. The invention also provides a patient interface which uses the cushion arrangement of the invention.

The cushion arrangement of the invention uses a shrink material to deform the cushion into a shape which corresponds better to the patient face. In this way, a default compression or expansion pattern is fixed into the cushion. The customisation can thus simply involve a heating process, which can be carried out by the clinician, for example in a sleep lab. This enables a reduction of cost in order achieve the desired increase in patient comfort.

There may be a choice of starting cushions or patient interface devices (i.e. masks). Thus, the customizable cushion may come in a number of standard sizes (for example 2 or 3) which can be adjusted using a simple technology. The starting cushion or patient interface device can if needed be used directly without customization as a standard patient interface device, for example if it is not to be worn for a long period of time, or if it is already a comfortable fit.

The cushion arrangement and patient interface device of the invention can be mass produced and the customization can be carried out directly in a sleep lab using a simple heating tool.

The shaping structure can comprise a band applied to the cushion, for example around an outer edge of the cushion.

The shaping structure can comprise a band of shrink elements, wherein the amount of shrinkage applied to each shrink element is individually selected. These shrink elements are then positioned around the cushion which is annular, and each one performs a local positioning function. There may by 4 to 100 individual shrink elements. This avoids the shaping layer adding too much rigidity to the cushion.

In the method of the invention, the local dimension of the themo-shrink material determines is controllled to define a level of local compression or expansion of the mask cushion, and in combination these correspond to the patient face more closely.

The deforming of the shaping structure can provide the forces needed to move the cushion into the deformed (locally compressed or expanded) state.

Instead, the method can further comprise mechanically holding the cushion in the compressed state before applying the heating. The heating then performs the shrinkage or expansion to an amount which depends on the mechanical position previously held.

The method can comprise applying heat to one location, and rotating the cushion arrangement so that heat is applied all around the shaping structure, wherein the duration of the heating at different points around the band is controlled to implement the selected amount of shrinkage to individual shrink elements. This provides a simple way to implement fully customisable shaping.

The cushion arrangement used as the starting point can be selected as one of a set of default cushion arrangements which is the closest fit to the patient.

This apparatus of the invention can be provided in a clinician's office for use in customising a mask for a specific patient.

### BRIEF DESCRIPTION OF THE DRAWINGS

Examples of the invention will now be described in detail with reference to the accompanying drawings, in which:

FIG. 1 shows a known patient interface;

FIG. 2 shows in simplified from a patient interface of the invention;

FIG. 3 shows a first way of implementing cushion shaping in accordance with the invention;

FIG. 4 shows an example of apparatus for implementing the heating;

FIG. 5 shows a second way of implementing cushion shaping in accordance with the invention;

FIG. 6 shows a second example of arrangement of the thermo-shrink band;

FIG. 7 shows an example of apparatus for applying a mechanical bias;

FIG. 8 shows a third way of implementing cushion shaping in accordance with the invention;

FIG. 9 shows a fourth way of implementing cushion shaping in accordance with the invention;

FIG. 10 shows a fifth way of implementing cushion shaping in accordance with the invention;

FIG. 11 shows a sixth way of implementing cushion shaping in accordance with the invention;

FIG. 12 shows a seventh way of implementing cushion shaping in accordance with the invention;

FIG. 13 shows how the heating is carried out for the example of FIG. 12; and

FIG. 14 shows an eighth way of implementing cushion shaping in accordance with the invention.

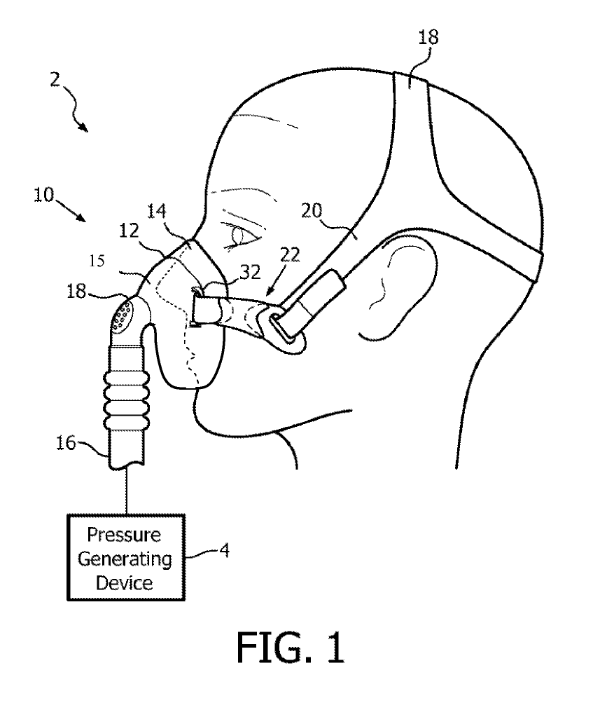

### DETAILED DESCRIPTION OF THE EMBODIMENTS

The invention provides a cushion arrangement for a patient interface for communicating with the nose or the nose and mouth of a patient, and it provides the patient interface using the cushion. A shaping structure is in contact with the cushion. The shaping structure comprises a thermo-shrink material which can be customised for the end user, and it applies a pre-compression or an expansion to the cushion.

The difference between the shape of the face of a user and the cushion geometry results either in discomfort or else it results in air leakages, or both. By pushing the patient interface device (i.e. the mask part) very tightly on the face, the air leakages can be eliminated at the cost of high pressure points, a process resulting in red marks and, therefore, decreasing the patient/mask compliance.

The aim of the invention is to reduce the pressure created on the face by using a customisation process which means the pressure holding function is at least partly obtained by a shaping layer made of thermo-shrinking material.

The customizable cushion (and corresponding patient interface using the same) described below allows a mass-production of all parts, and the actual customization is limited to the special cushion for the patient interface, i.e. the mask cushion. A mass produced mask cushion can be made with over-sized height, and then the height is reduced in a controlled and shaped manner (if needed), by means of the heat-shrinking shaping layer.

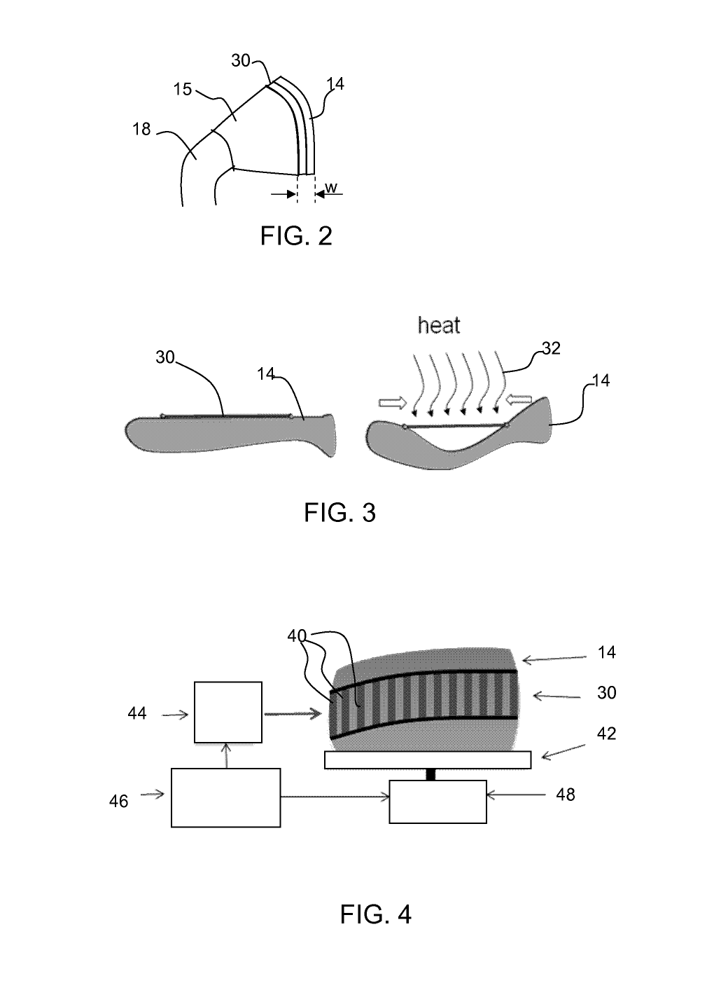

FIG. 2 shows in conceptual form the patient interface of the invention in the form of a mask. Only the mask shell 15 and mask cushion 14 are shown, since the invention only relates to these components.

The invention provides a shaping structure 30 in contact with the mask cushion 14 and which comprises a thermo-shrink material. In the first set of examples, the shaping structure is a shaping layer applied to the cushion.

The shaping layer is applied across at least a portion of the width (shown as w in FIG. 2) of the cushion, where this width is the direction along which the cushion is to be compressed, i.e. the direction in which force is applied against the face by the straps, and basically perpendicular to the plane of the face of the patient. The shaping layer is used to implement a compression of the cushion 14 in the manner of a fixed pre-bias.

In this example, the shaping layer is oriented in such a way that it shrinks in a direction orthogonal to the cushion perimeter, and not along the perimeter (i.e. in the width direction). The shaping layer 30 can be an integral part of the cushion 14.

Two customization approaches possible. If the shaping layer 30 is thick enough (in the width direction) then it is also strong enough to compress the cushion wall by itself without any external force. This is shown in FIG. 3.

FIG. 3 shows a cross-section of the mask cushion 14 with the thermo-shrinking layer in the form of a band around the cushion outer wall. The band 30 deforms the cushion ring without any external force, when heat 32 is applied.

FIG. 4 shows a hot air heat tool which can be used to control the heat shrinking.

The shaping layer 30 is shown as a series of parallel shrink elements 40 around the outside of the cushion 14. These elements 40 extend in the width direction, namely the direction in which the cushion is compressed in use. They form a ladder around the outside of the cushion 14.

The cushion (or mask with cushion attached) is placed on a rotary table 42. There is a single heating location, where heat is directed by a heater 44 in the form of a laser or warm air generator. A controller 46 controls a motor 48 which governs the rotation of the table 42 as well as controlling the heater 44.

The individual shrink elements perform a local compression of the cushion. The amount of deformation is controlled by controlling the treat time and/or the level of heating applied. In one example, the heating applied is constant, and the duration is controlled by increasing and decreasing the speed of rotation. The rotation can be continuous with variable speed or it can be stepped.

By designing the band as a series of shrink elements, the stiffness of the shaping band is reduced, so that additional deformation of the mask cushion under load is still possible. A continuous band of shaping material may increase the stiffness too much. The heating apparatus of FIG. 4 allows direct customization in a sleep lab for example.

If the mask is already a good fit, or if the comfort is less important (because it will not be worn for a prolonged period) the mask can be used directly without customization.

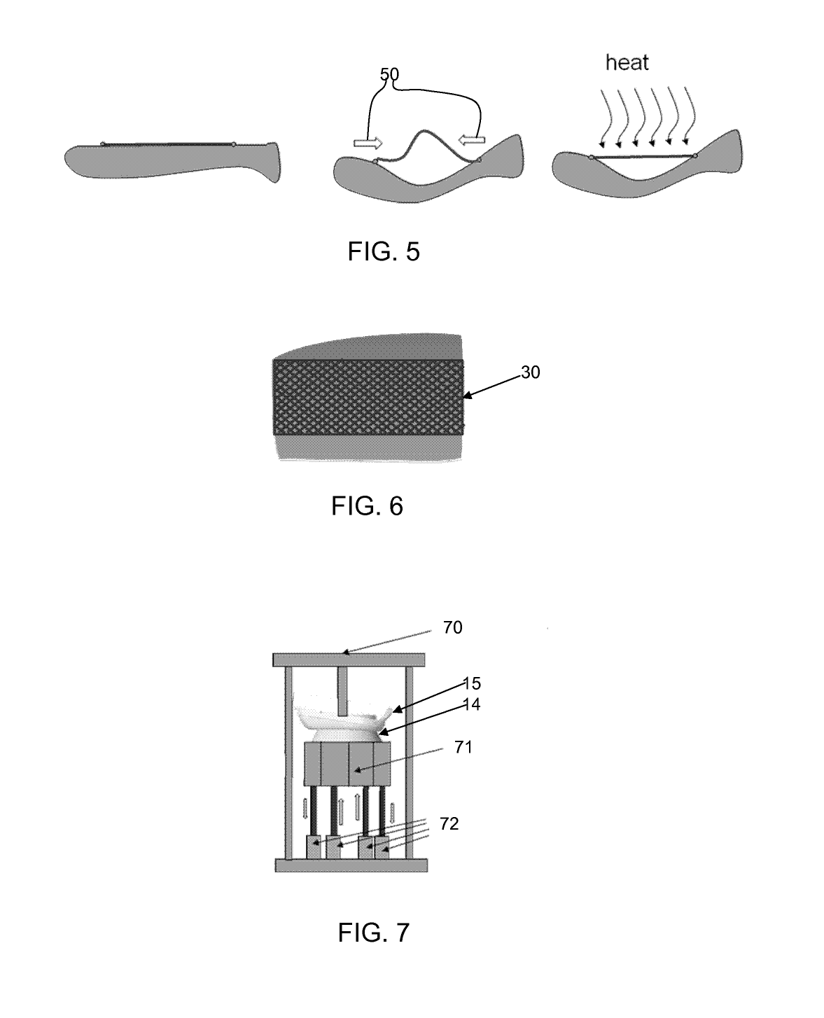

If the shaping layer is thin, then it may not apply the necessary force during shrinking to deform the cushion. In this case, a mechanical preload can be applied before the shrinking as shown in FIG. 5. A relatively thin shaping layer can however prevent the compressed cushion from returning to its original state.

FIG. 5 shows customization by fixing the deformation obtained by application of an external force 50 before the heat treatment.

The shaping band can be integrated on the outer side of the cushion and fixed at the top and the bottom edges by means of gluing or overmolding.

The shaping band can be glued with a UV-curable polymer, in order to keep the temperature of the band below the critical point at which the shrinking process begins.

By integrating the thermo-shrinking shaping band on the outer side of the cushion, it is easier to reach for manipulation, and the air outside the cushion is less humid and therefore less bacteria will grow behind the band. The requirements for the chemical stability/neutrality of the materials on the outer side of the cushion are naturally less restrictive.

In order to further reduce dependency between the adjacent parts of the cushion perimeter and improve mechanical properties of the cushion, the heat-shrinking band can be in the form of a perforated sheet as shown in FIG. 6.

FIG. 7 shows an apparatus 70 for performing the mechanical preload mentioned with reference to FIG. 5.

The apparatus is loaded with the cushion 14 which sits on a multi-segment tray 71. Under each segment there is a motor 72 which allows the local adjusting the height of the tray segments and, therefore, the local cushion compression.

The cushion manipulators can move slightly diagonally instead of purely perpendicularly. After the mask is pressed between the mask holder and the tray, the cushion is heat-treated to fix the achieved deformation.

In order to control the customisation process, the contour of the patient's face needs to be determined, and used as an input to the controller 46 in FIG. 4.

This contour measuring process can be implemented in a number of different ways.

By way of example, a contact or contactless facial scanner can be used, e.g. a 3D structured light scanner which outputs a 3D head model.

A processing module can then be used to detects the locations of facial landmarks of the 3D head model, such as nose top, mouth corners, nose corners, eye corners, chin deep, etc. A 2D mask perimeter contour can then be aligned with respect to the detected landmarks using pre-defined mask fitting rules. For example, the mask 2D contour can be aligned symmetrically with respect to the face such that it passes through the deepest point between the lower lip and the chin.

A processing module can then be used to project the aligned 2D mask perimeter contour onto the face to derive a 3D facial contour. This 3D facial contour can then be compared with a default mask 3D contour, and local differences can then be computed. These differences are then translated into the required local mask deformations.

For this purpose the formula: S=a*D+b can be used, where S is the required amount of local shrinkage of the mask cushion, D is the local difference between the found 3D facial contour and the default 3D mask contour, and a,b>0 are parameters.

The input controller 46 uses the the amount of local shrinkage S as input parameter to control the heater 44.

The shaping layer can comprise the shrink elements applied to a backing layer which is then bonded to the mask cushion 14, or else the shrink elements can be bonded directly to the mask cushion.

A limited number of adjustments around the cushion perimeter are needed, since the cushion will naturally adopt a smooth profile between those points. There may be 4 to 100 points around the cushion perimeter where the width is controlled by the shaping band 30.

Each shrink element may be for example 5 mm to 40 mm long and have a width of 1 mm to 5 mm and thickness of 0.03 to 0.5 mm depending on the function (restraining or compressing the cushion).

Even if a continuous shaping band is used, as in FIG. 6, the heating may result in a discrete set of points where the thermo-shrinking is controlled.

Known materials are available for the thermo-shrinking material, for example materials used in shrink wrap packaging. These are typically polymer plastic films. When heat is applied they shrink tightly over whatever is being covered. Heat can be applied with a hot air gun. The most commonly used shrink wrap is polyolefin. It is available in a variety of thicknesses, clarities, strengths and shrink-ratios. An activation temperature above 100 degrees Celsius prevents shrinkage at normal temperatures. The material is bio compatible and it is widely used in food industry.

Other suitable thermo-shrinking materials will be known to those skilled in the art.

The various examples above use the thermo-shrinking to implement a controlled local compression of the cushion. It is instead possible to implement a local expansion using a shrinkage. There are various possible ways to achieve this.

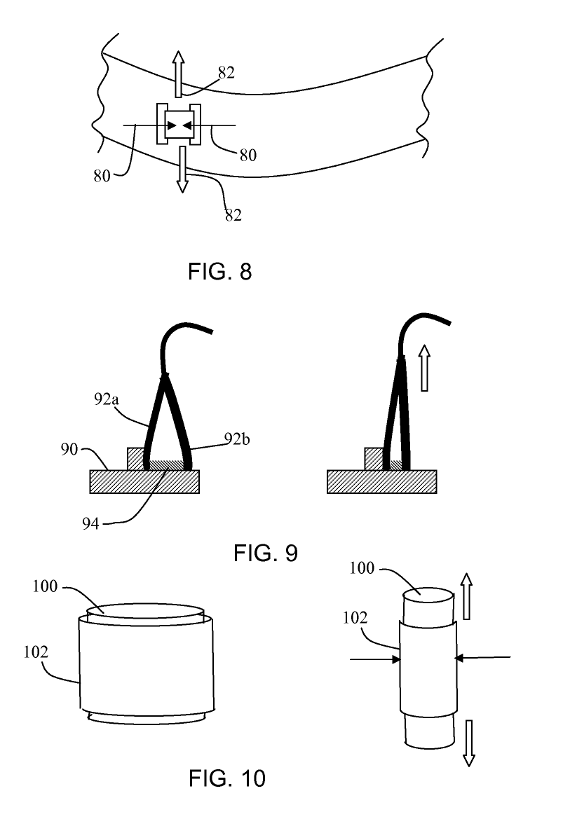

FIG. 8 shows how a thermo-shrink element can be arranged so that the shrinkage takes place in a circumferential direction around the perimeter of the cushion. By fixing the ends of the element to the cushion 14, when the shrinkage takes place, there is bulging of the cushion in the axial direction, i.e. the thickness direction of the cushion. The thermo-shrinkage is shown as arrows 80, and the resulting cushion expansion is shown as arrows 82.

Again, individual shrink elements can be heated and the shrink elements are part of a layer applied to the cushion perimeter.

FIG. 9 shows another way to convert shrinkage to cushion expansion. FIG. 9 represents a cross section of a side wall of the cushion, i.e. a slice radial slice assuming an annular cushion shape. The cushion is formed on a rigid base 90, and has thicker flexible sections 92a, 92b, such as silicone, between the rigid base 90 and a thinner flexible section 96 which adapts to the shape of the user's face. The thicker flexible sections are sufficiently rigid to maintain their length but sufficiently flexible to be movable.

The more rigid sections are used for shape control, and they define a triangle, formed as two sides 92a,92b of the thicker material, and one side 94 of the thermo-shrink material. When the side 94 contracts, the triangle height increases, given the constant length of the sides. This is shown as arrow 98.

Again, different areas around the cushion perimeter can be heated differently.

FIG. 10 shows another way to convert shrinkage to length increase, which can be used to expand the cushion locally.

The thermo-shrink material is formed as a band 102 around a shaft 100 of flexible material, such a silicone. The band contracts radially when heated, causing bulging of the shaft, as shown in the right image of FIG. 10.

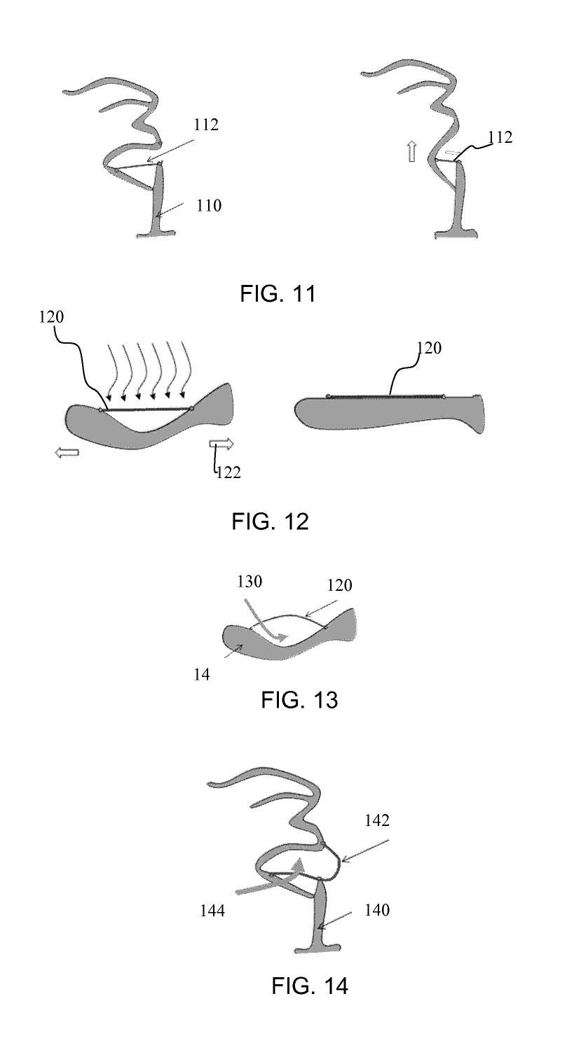

FIG. 11 shows another example. A radial cross section of the cushion is again shown. The cushion has a collapsed state in the left image, and it is pulled into an upright state by shrinkage of a connecting rod or band 112 between two parts of the cushion structure 110.

The thermo-shrinkage will only take place if the shrinkage force overcomes the forces restraining the material. Similarly, if the thermo-shrinkage material is under a tensile load, when heating is applied, the material can expand under the existing tensile load, i.e. be stretched when the material properties have changed through heating.

This gives another set of examples of how to use the thermally induced change in the material to alter the cushion shape.

FIG. 12 shows a first example.

The cushion has a thermo-shrinking band 120 which retains a compressed groove, and is therefore under tensile load applied by the cushion. When the restraining properties of the band 120 are relaxed by heating, the cushion expands. However, it will only expand when free to do so. Thus, if the cushion is constrained to a certain shape, the degree of relaxation which can take place will match the cushion shape.

This design can be customized directly on the face.

The cushion is pressed to a patient face which will have non-complying geometry. Areas with different pressure characteristics will be created along the cushion perimeter. In some areas, the cushion may expand before it reaches the face of the patient, and in others it may be held in an even further compressed state.

When the thermo-shrinking band is heated, for example by circulating hot air 130 in the tunnel formed by the grove and the band as shown in FIG. 13, then the groves will open up in the areas with insufficient pressure and the further restrained in the areas with excessive pressure. After the band is cooled, the cushion will keep the new shape. The heating in this case does not need to be controlled to provide the desired local degree of shrinkage, because it depends on the load applied to the cushion. Instead, warm air is circulated all around the perimeter.

It can be seen that this example combines shrinkage and expansion of the thermo-shrink material upon heating, depending on the load existing on the material when heated. The cushion expansion is effected as a release of a stored tensile load.

A further example is shown in FIG. 14. This is similar to the example of FIG. 11 in that the length of the thermo-shrinkage band 142 is a function of a degree of collapse of the cushion. The mask is shown as 140. The shape setting can again be controlled by uniform heating all around the mask periphery with the mask applied to the user's face.

When the air is blown in the tunnel 144 between the band 142 and the cushion 140, the air pressure lifts the cushion in the places with insufficient facial pressure. In the places with excessive facial pressure the cushion is compressed. Under the action of the hot air the thermo-shrinking band shrinks fixing the cushion shape. Thus, in this case, the cushion expansion is caused by pressure of the heating air, and this is then locked in place.

It is therefore clear from the examples above that the change in properties of a thermo-shrink material can be used in various ways to implement a controllable expansion or compression of the cushion to a desired shape. In cases where a shape is applied to the cushion, the heating does not need to be locally selective and the heating can take place with the mask applied to a user.

The invention can be embodied as a cushion alone, which is supplied separately to the rest of the patient interface device, or it can be embodied as a patient interface device (i.e. a mask), or as a full system.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion arrangement for a patient interface (10) for communicating with the nose or the nose and mouth of a patient, comprising a cushion (14) and a shaping structure (30) in contact with the cushion (14), characterized in that the shaping structure comprises a thermo-shrink material, and the local dimension of the themo-shrink material determines a level of local compression or expansion of the cushion.

2. A cushion arrangement as claimed in claim 1, wherein the shaping structure (30) comprises a band applied to the cushion (14).

3. A cushion arrangement as claimed in claim 2, wherein the shaping structure (30) comprises a band applied around the outside of an outer edge of the cushion.

4. A cushion arrangement as claimed in claim 2, wherein the shaping structure comprises a band of parallel shrink elements

5. A patient interface (10) for communicating with the nose or the nose and mouth of a patient comprising a shell (15) and a cushion arrangement as claimed in claim 1.

6. A method of customising a cushion arrangement for a patient interface (10), comprising:
providing a cushion arrangement comprising a cushion (14) and a shaping structure (30) in contact with the cushion (14), wherein the shaping structure (30) comprises a thermo-shrink material;
applying heat to the shaping structure (30) thereby to permanently deform the shaping layer as a function of the face shape of the patient, and thereby to hold the cushion (14) in a compressed or expanded state which corresponds more closely to the face shape.

7. A method as claimed in claim 6, comprising using the deforming of the shaping structure (30) to move the cushion into the compressed or expanded state without externally holding the cushion in the compressed or expanded state.

8. A method as claimed in claim 6, further comprising mechanically holding the cushion (14) in the desired state before applying the heating.

9. A method as claimed in claim 8, wherein mechanically holding the cushion (14) in the desired state comprises applying the cushion against the face of a user.

10. A method as claimed in claim 6, wherein the shaping structure (30) comprises a band of shrink elements (40), wherein the method comprises applying an individually selected amount of shrinkage to each shrink element.

11. A method as claimed in claim 10, comprising applying heat to one location, and rotating the cushion so that heat is applied all around the band (30), wherein the duration of the heating at different points around the band (30) is controlled to implement the selected amount of shrinkage to each shrink element (40).

12. A method as claimed in claim 6, wherein providing a cushion arrangement comprises selecting the one of a set of default cushion arrangements which is the closest fit to the patient.

13. A method as claimed in claim 6, further comprising analysing the face of the patient to derive the desired cushion shape, and using this to control the heating.

14. An apparatus for customising a cushion arrangement of a patient interface (10) for communicating with the nose or the nose and mouth of a patient, comprising:
a support (42) for the cushion arrangement, the cushion arrangement comprising a cushion (14) and a shaping structure (30) in contact with the cushion, wherein the shaping structure (30) comprises a thermo-shrink material;
a heater (44) for applying heat to the shaping structure (30) thereby to permanently deform the shaping structure as a function of the face shape of the patient, and thereby to hold the cushion (14) in a compressed state which corresponds more closely to the face shape.

15. An apparatus as claimed in claim 14, wherein the support (42) comprises a rotary table.
